(19) United States
(12) Patent Application Publication
(10) Pub. No.: US 2009/0104157 A1
(43) Pub. Date: Apr. 23, 2009

(54) UTILIZATION OF BACTERIOPHAGE TO CONTROL BACTERIAL CONTAMINATION IN FERMENTATION PROCESSES

(75) Inventors: Ethan Baruch Solomon, Wilmington, DE (US); Derrick Okull, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. du Pont de Nemours and Company, Wilmington, DE (US)

(21) Appl. No.: 12/245,272

(22) Filed: Oct. 3, 2008

Related U.S. Application Data
(60) Provisional application No. 60/997,809, filed on Oct. 5, 2007.

(51) Int. Cl. A61K 35/76

(52) U.S. Cl. 424/93.6

(57) ABSTRACT

A fermentation process for the production of ethanol from natural sources, such as corn, comprising introducing a fermentable sugar and an inoculant, and a bacteriophage cocktail into a fermentation system and introducing a cocktail comprising one or more lytic bacteriophage is added to one or more of the fermentable sugar, the inoculant, or the fermentation system. Bacteriophages that infect and lyse the bacteria that contaminate fermentable sugars are selected. The bacteriophage cocktail is added in an amount effective to substantially prevent growth of these bacteria.

### CROSS REFERENCE TO RELATED APPLICATION

This application claims priority under 35 U.S.C. § 119 from U.S. Provisional Application Ser. No. 60/997,809 (filed Oct. 5, 2007) which is incorporated by reference herein for all purposes as if fully set forth.

### FIELD OF THE INVENTION

The present invention relates to controlling populations of contaminating bacteria that decrease the yield of fermentation processes. This invention is useful for industrial/commercial fermentations in which yeasts, bacteria, or fungi are used to produce an end product.

### BACKGROUND OF THE INVENTION

Fermentation processes are used to produce a wide variety of commercial products such as ethanol, antibiotics, enzymes, and other microbial metabolites. In these processes, yeasts, bacteria, or fungi convert an incoming feedstock into a desirable end product. Feedstocks used in industrial fermentations normally provide both an energy source to the fermenting organism and precursors for conversion into the end product.

Feedstocks used in commercial fermentations vary greatly. Certain fermentation products such as vitamins, amino acids, hormones, and enzymes are produced using carefully controlled feedstocks under aseptic conditions. Other fermentation products are produced using raw natural materials and municipal or crop wastes as feedstocks under far less controlled conditions. The feedstocks for these “other” fermentation products may be contaminated with microorganisms which compete with yeast, bacteria or fungi for nutrients, and convert feedstock reactants to undesirable byproducts. Growth and competition for nutrients by contaminating microorganisms are significant concerns when using raw natural materials or wastes as fermentation feedstocks.

Ethanol is a major chemical product which man has produced by fermentation for millennia from a feedstock which has a natural source. Currently ethanol is produced at large scale from natural sources by fermentation of sugar to ethanol and carbon dioxide in the presence of yeast. Many feedstocks can be used to provide the sugar for fermenting. Current raw natural materials include corn, milo, wheat, barley, millet, straw, sorghum, sugar cane, sugar beets, molasses, whey, and potatoes. In fact, any starch or cellulosic material, which includes nearly all plants, can be used as a natural source of sugar for use in producing ethanol, as starch or cellulose is a precursor to sugar.

Given that ethanol fermentation uses raw natural materials as a feedstock, microbial contamination by (competing) bacteria is a grave concern. The yeasts used to produce ethanol are forced to compete with these contaminating bacteria for sugars in the feedstock. A wide variety of unwanted contaminating bacteria can be carried into the fermentation by the feedstock. Of primary concern are Lactobacillus, Lactococcus, Pediococcus, Leuconostoc, Enterococcus, Weissella, Oenococcus, and Bacillus, among other species commonly present in this type of environment. Unfortunately, the optimum atmosphere for ethanol fermentation is also extremely conducive to bacterial growth. Contaminating bacteria convert sugar (glucose) to organic acids, such as acetic acid and lactic acid, rather than ethanol. Furthermore, bacteria grow rapidly in the nutrient rich environment of a fermentation system, and consume sugar (glucose) faster than does yeast. Organic acids produced by the bacteria inhibit performance and growth of the yeast. Thus, bacterial infection results in decreased yield of ethanol, and the fermentation process becomes less economical.

Current industry strategies to combat bacterial infection in fermentation systems include monitoring for the presence of organic acids (e.g., acetic acid and lactic acid) followed by remedial treatment with antibiotics or biocides to control bacterial growth. However, this method permits high levels of bacterial growth to occur before treatment and may require comparatively high levels of antibiotics or biocides.

The use of antibiotics to reduce bacterial growth in fermentation systems has become disfavored. Antibiotics can remain and accumulate in solid products of fermentation, since they do not become deactivated upon reaction with target bacteria. Solid products include distillers dried grain solids (DDGS) and distillers wet grain solids (DWGS). DDGS and DWGS are valuable byproducts of fermentation and are used in animal feeds. In many countries the amount of antibiotics in animal feed is under or being considered for regulatory control. Bacteria are also becoming resistant to the antibiotics commonly used in fermentations.

Biocides have also been used as remedial treatments for fermentation systems. Generally, biocides perform poorly because they are non-specific and attack yeasts as well as the contaminating bacteria.

An alternative to remedial treatment is to proactively prevent growth of contaminating bacteria. Addition of antibiotics in amounts to prevent growth of bacteria has been considered. However, the issue of antibiotics accumulating in fermentation solids remains.

There is a need to prevent and/or control growth of contaminating bacteria in fermentation processes, while minimizing or eliminating the use of antibiotics or biocides. The present invention meets this need.

### SUMMARY OF THE INVENTION

The present invention provides a process to substantially prevent the growth of bacteria in a fermentation system comprising introducing a fermentable material comprising a fermentable sugar and an inoculant into a fermentation system and introducing a cocktail comprising one or more lytic bacteriophage is added to one or more of the fermentable material, the inoculant, or the fermentation system wherein one or more lytic bacteriophage is active against lactic acid bacteria and wherein the cocktail is added in an effective amount at acid concentrations in the fermentation system of acetic acid no greater than 0.30% (weight/volume) and lactic acid no greater than 0.80% (weight/volume). Lactic acid bacteria include Lactobacillus, Lactococcus, Enterococcus, Weissella, Leuconostoc, Pediococcus, Streptococcus, and Oenococcus.

The fermentable material may be derived from a grain-based product such as corn, wood chips, wheat straw, corn stover, switch grass, and combinations of two or more thereof. The fermentable material may alternatively be derived from a feedstock selected from the group consisting of milo, barley, millet, sorghum, sugar cane, sugar beets, molasses, whey, potatoes, and combinations of two or more thereof. Preferably the fermentable material is derived from corn.

The initial density (amount) of bacteriophage introduced into the system may vary, but an effective amount is based on the ratio of bacteriophage plaque forming units (PFU) to bacterial colony forming units (CFU). This ratio is preferably of a magnitude at least 10 times (10×) the number of bacteriophage to the bacterial population in the fermentation system, more preferably, the ratio is at least 100× the bacterial population in the fermentation material. When the fermentable material is derived from corn, a ratio of 10 bacteriophage plaque forming units to 1 bacterial colony forming unit corresponds 10⁴ plaque forming units of bacteriophage per gram of fermentation material.

### BRIEF DESCRIPTION OF THE DRAWING

FIG. 1 is a bar graph illustrating presence of Lactobacillus plantarum in an culture with and without treatments in accordance with Example 1.

### DETAILED DESCRIPTION OF THE INVENTION

The present invention relates to industrial fermentation processes in which there is a need to control contaminating bacterial populations in a feedstock. For example, during the production of ethanol via fermentation, contamination by lactic acid bacteria (LAB) can negatively impact the production of ethanol by yeast. In this invention, a cocktail comprising lytic bacteriophage for LAB is introduced into a fermentation system that is susceptible to bacterial contamination. Lactic acid bacteria that may be present in a fermentation system are Lactobacillus, Lactococcus, Enterococcus, Weissella, Leuconostoc, Pediococcus, Streptococcus, Oenococcus and combinations of two or more thereof.

Bacteriophages (phages) as used herein refer to viruses that utilize bacteria as their hosts. Lytic bacteriophages utilize the host's metabolic machinery to produce copies of the phage DNA and new viral particles, to assemble progeny phage, and then to burst out of the host cells in a process known as “lysis.” Lysis results in the death of the original host cell and the release of large numbers of progeny phages which are free to act on new host bacterial cells.

Phages are relatively specific in their ability to infect host bacteria, and act on bacteria only, with no ability to infect yeasts (eukaryotes). In this invention, bacteriophages that specifically lyse LAB are used to control and/or reduce the growth of LAB and minimize their effect on a fermentation process.

The following is a description of how the process of this invention may be performed in ethanol fermentation using corn as the feedstock. It will be understood by those skilled in the art this process may be varied. It will be further understood that this invention can be used in other fermentation processes wherein yeast, fungi or recombinant bacteria are used to produce an end product.

Ethanol can be produced from corn or other grain in a wet mill or a dry mill process. In a wet mill process, corn is soaked or steeped and then separated into components. In a dry mill process, corn is ground into meal and processed without separation. The corn starch component from the wet mill process or meal from the dry mill process is mixed with water and enzymes and cooked in a cooking tank to solubilize the starch, a process commonly referred to as liquefaction.

Corn starch is a polysaccharide, that is, a polymer, made of individual units of glucose. Starch is converted to smaller (shorter) polysaccharides, i.e., dextrins, by the enzyme α-amylase. The smaller polysaccharides are converted to a fermentable sugar, that is, glucose (monosaccharide), using the enzyme glucoamylase. The product resulting from enzyme treatments is a fermentable material comprising a fermentable sugar.

The process to produce ethanol then comprises fermenting the sugar in a batch or continuous reactor by contacting the fermentable material with an inoculant, such as yeast, in a fermentation system, to produce a fermentation product comprising ethanol and carbon dioxide. Subsequent steps include (1) distilling the fermentation product to remove about 95% of the water, as well as the solids to produce a distilled ethanol comprising about 5% water; and (2) dehydrating the distilled ethanol, thereby producing 100% (200 proof) ethanol. Additional steps comprise denaturing the dried ethanol by mixing in about 2-5% gasoline or other additive for non-liquor uses; and recovering co-produced carbon dioxide and solids. These steps are known to those skilled in the art.

Processes for the production of fuel ethanol are performed under conditions which do not preclude introduction of bacteria to fermentation systems. Sources of bacteria in a fermentation system may include any of the feeds (fermentable material, inoculant) introduced into the system. Inadequate cleaning between batches or runs may also be a source of bacteria for subsequent fermentations. Bacterial infections of fermentation systems produce byproduct organic acids, particularly acetic acid and lactic acid, which consume (i.e., react with) ingredients (fermentable sugar) and inhibit activity of the inoculant. Acetic acid and lactic acids are also produced by yeast during fermentation, but in amounts not sufficient to significantly interfere with the overall yield and efficiency of the process. Increasing concentrations of the organic acids suggest growth of bacteria in a fermentation system.

In the process of the present invention, a cocktail comprising one or more lytic bacteriophage is added to one or more of the fermentable material, the inoculant, or the fermentation system, in an amount effective to infect and lyse (destroy) contaminating bacteria and thus inhibit the formation of high levels of organic acids in the system. That is, the bacteriophage cocktail is added to the system prior to the substantial growth of bacteria.

The bacteriophage to be used in these cocktails may be selected from a variety of sources. Phages specific for many different bacterial genera are available from repositories such as the American Type Culture Collection (Manassas, Va.). Bacteriophages may also be procured by isolation from natural sources that harbor high levels of the host bacteria. For example, using corn feedstocks for ethanol fermentation, phages may be isolated from incoming corn, milled corn, steeped corn, slurry, or from within the fermenter itself. Additional sources may include sewage, animal and plant waste materials and estuary water. Phage isolation involves incubating (enriching) samples containing phage with potential bacterial hosts. These procedures are well known to those skilled in the art.

In order to substantially prevent growth of bacteria and significant formation of organic acids in a fermentation system, a cocktail comprising one or more bacteriophage wherein the bacteriophage is selected from one or more lytic phage active against lactic acid bacteria is added to one or more of the fermentable sugar, the inoculant, or the fermentation system. For example, lactic acid bacteria include Lactobacillus, Lactococcus, Enterococcus, Weissella, Leuconostoc, Pediococcus, Streptococcus, and Oenococcus.

It is generally known that populations of these bacteria in a typical fermentation process wherein the fermentable material is derived from corn feedstock number at least 10³ colony forming units (CFU) per gram (see, for example, Skinner and Leathers, “Bacterial contaminants of fuel ethanol production”, J. Ind. Microbiol. Biotechnol. (2004), 31: 401-408). The natural occurrence of bacteria in other feedstocks can be similarly determined. The cocktail of lytic phage is added to the fermentable material, the inoculant (e.g., in the yeast propagation tank), or the fermenter itself. The initial density of bacteriophage introduced into the system may vary, but is based on the ratio of bacteriophage plaque forming units (PFU) to bacterial colony forming units (CFU). This ratio is preferably of a magnitude at least 10 times (10×) the bacterial population in the fermentation system (10 bacteriophage PFU to 1 bacterial CFU), or at least 10⁴ plaque forming units per gram of fermentation material when the fermentable material is derived from corn. More preferably, the ratio is at least 100× the bacterial population (100 bacteriophage PFU to 1 bacterial CFU) in the fermentation material. It will be understood by those skilled in the art that increasing the ratio of bacteriophage to bacterial cells beyond this magnitude will result in more effective bacterial control. One benefit of the use of a lytic phage cocktail is the ability of one phage to produce a large number of progeny phages. This allows the continuous control of target bacteria as progeny phages are produced.

### Fermentable Material

A fermentable material comprising a fermentable sugar suitable for use in this invention can be derived from a raw natural material as a feedstock. Essentially any plant source comprising sugar, starch and/or cellulose can be used as a feedstock. That is, starch and/or cellulose can be converted by processes known in the art, e.g., using enzymes, to sugar suitable for use as a fermentable sugar in this invention. The fermentable sugar can be derived from a feedstock selected from the group consisting of grain-based product such as corn, wood chips, wheat straw, corn stover, switch grass, and combinations of two or more thereof. The fermentable sugar may alternatively be derived from a feedstock selected from the group consisting of milo, barley, millet, sorghum, sugar cane, sugar beets, molasses, whey, potatoes, and combinations of two or more thereof. Processes are known to those skilled in the art to convert these materials to a fermentable material.

Conveniently and preferably, the fermentable material is derived from corn. The corn feedstock may undergo either a wet mill or a dry mill process, followed by liquefaction to produce a liquefied starch. The liquefied starch undergoes saccharification, a process in which the starch is contacted with enzymes, to convert the starch to glucose, thus forming the fermentable sugar in the fermentable material.

In a fermentation process, sugar is typically present at a concentration of about 5 to about 40% (weight/volume), preferably in the range of about 10 to 30% (weight/volume) in the fermentable material.

### Inoculant

For purposes herein, an inoculant is a microorganism which is capable of converting a fermentable sugar to ethanol. Yeasts are common inoculants, which are used in ethanol fermentation. Yeasts are microorganisms capable of living and growing in either aerobic (with oxygen) or anaerobic (lacking oxygen) environments.

The following discussion is directed to a process in which the inoculant is yeast.

Relative to bacteria, yeasts may have moderate to slow fermentation rates. To compensate for their metabolic rate, large amounts of yeast may be required in large scale industrial ethanol production. Typical holdup times for the fermenting step when using yeast are 40-72 hours. Prior to introducing yeast into a fermenting vessel, a yeast inoculum is produced in a yeast propagation tank separate from the fermenting vessel. A yeast inoculant is produced, for example, from dry yeast in the propagation tank. In a propagation tank, a yeast starter culture is supplied with nutrient composition, which may comprise fermentable sugar, enzymes, and water to activate or grow the yeast. Yeast propagation also occurs during the fermenting step. However, activation of yeast in a propagation tank provides highly active yeast upon introduction to the fermenting vessel.

Inoculant yeast is added to the fermentation system in an amount to produce 10⁶ to 10⁸ yeast cells per milliliter of mash. It will be recognized by those skilled in the art that this amount may vary.

### Bacteriophage

The term bacteriophage (or phage) means any virus that infects bacteria. More specifically, the term bacteriophage as used herein refers to viruses that lyse and destroy their hosts (lytic bacteriophages).

Ethanol fermentation process is generally affected by contamination by members of the Lactic Acid Bacteria (LAB). LAB are naturally present in corn and other agricultural products in which they play an important role in the conversion of the raw products into a palatable form for animal feed (ensilation). During ethanol fermentation, growth of LAB is not desired due to the competition for sugars and production of organic acids. Consumption of sugar and production of organic acids (e.g., acetic acid and lactic acid) by the LAB reduces the efficiency with which fermenting yeasts convert the sugar to ethanol, thus decreasing final yield. Therefore, it is desirable to use bacteriophage that target LAB to decrease the levels of LAB in the corn feedstock.

Bacteriophages are ubiquitous in nature. Bacteriophages can be isolated from a number of sources. Sources include corn (milled, steeped, and/or in slurry form, for example), sewage, animal and plant waste materials, and estuary water. Bacteriophages can be isolated using known bacteriophage enhancement techniques, from the bacteriophage source and are available from known repositories.

The bacteriophage cocktail as described herein comprises one or more bacteriophage specific for genera of LAB commonly found in feedstocks, especially corn, used for ethanol production. The bacteriophage cocktail comprises one or more bacteriophage effective for lysis of LAB, such as Lactobacillus, Lactococcus, Enterococcus, Weissella, Leuconostoc, Pediococcus, Streptococcus, Oenococcus and combinations of two or more thereof. In addition, the cocktail may comprise a newly isolated phage specific for any of the above genera. The bacteriophage cocktail is generally provided as a liquid suspension in an aqueous buffer.

The bacteriophage is present in the fermentation system in an amount of at least 10 phage to 1 bacteria cell. The bacteriophage can be used in an amount as high as 10,000 phage per bacteria cell. Preferably, the bacteriophage is present in an amount of at least 100 phage to 1 bacteria cell, more preferably at least 1000 phage to 1 bacteria cell. While it is recognized that higher amounts of bacteriophage will generally increase effectiveness, practical considerations such as cost and overall efficiency of the process may be used to determine appropriate loading of bacteriophage.

### Process

The present invention is a process to substantially prevent the growth of bacteria in a fermentation system comprising introducing a fermentable material comprising a fermentable sugar, an inoculant, and a bacteriophage cocktail into the system wherein the cocktail comprises one or more lytic bacteriophage active against lactic acid bacteria. The bacteriophage cocktail can be added at any point in the fermentation process. As a preventative, to substantially reduce and/or prevent growth of lactic acid bacteria, the bacteriophage cocktail is added in an effective amount at acid concentrations in the fermentation system of acetic acid no greater than 0.30% (weight/volume) and lactic acid no greater than 0.80% (weight/volume). Alternatively, the phage cocktail is added as a preventative when bacterial populations are no more than 10³ CFU per gram of fermentation material. As the population of bacteria present in the process increases, so will the initial density of the bacteriophage required in order to maintain the minimum ratios and achieve complete control of the bacteria. It is known to those skilled in the art that acetic acid and lactic acid may be present in small amounts in a fermentation system, that is, without substantial bacterial growth. These organic acids can form as a byproduct of yeast fermentation of sugar.

Low concentrations of acetic and lactic acids are indicative of low levels of bacteria in the fermentation system. Thus, the level of bacterial contamination can be monitored by measuring acid concentration, i.e., of acetic acid and/or lactic acid. To effectively control bacterial growth, the bacteriophage cocktail is added in an effective amount. An effective amount of the bacteriophage is generally at least 10 phage to 1 bacteria cell (10 bacteriophage plaque forming units to 1 bacterial CFU.). The bacteriophage can be used in an amount as high as 10,000 phage or higher per bacteria cell (10,000 bacteriophage plaque forming units to 1 bacterial CFU.). Preferably, the bacteriophage is present in an amount of at least 100 phage to 1 bacteria cell (100 bacteriophage plaque forming units to 1 bacterial CFU.), more preferably at least 1000 phage to 1 bacteria cell (1000 bacteriophage plaque forming units to 1 bacterial CFU.). While it is recognized that higher amounts of bacteriophage will generally increase effectiveness, practical considerations such as cost and overall efficiency of the process may be used to determine appropriate amount of bacteriophage cocktail to add.

The bacteriophage cocktail comprises one or more bacteriophage lytic for lactic acid bacteria (LAB). LAB that may be present in a fermentation system are Lactobacillus, Lactococcus, Enterococcus, Weissella, Leuconostoc, Pediococcus, Streptococcus, Oenococcus and combinations of two or more thereof.

The bacteriophage cocktail may be added to the fermentable material or to the inoculant prior to their introduction into the fermentation system. By “fermentation system”, it is meant herein to refer to a batch or continuous flow fermentation tank or vessel or reactor (such as a plug flow reactor) in which the fermentation of sugar occurs. Alternatively or in addition, the bacteriophage may be added as a separate stream to the fermentation system, apart from the fermentable material and inoculant. It is recognized that bacteriophage may be added prior to or during fermentation, and if added prior to fermentation, after any processing steps that may cause deactivation of the bacteriophage, e.g., at a temperature greater than 50° C., such as liquefaction stage of ethanol fermentation of corn feedstock.

The bacteriophage cocktail is added in an amount effective to substantially prevent the growth of bacteria but have little impact on other variables in the fermentation process. This amount may vary, but is based on the ratio of bacteriophage plaque forming units to bacterial colony forming units. This ratio is preferably of a magnitude at least 10 times (10×) the bacterial population in the fermentation system, or 10⁴ plaque forming units per gram of fermentation material. More preferably, the ratio is at least 100× the bacterial population in the fermentation system. It will be understood by those skilled in the art that this amount may vary, but increasing the ratio of bacteriophage to bacterial cells beyond this magnitude will result in more effective bacterial control.

By operating a fermentation plant in accordance with this invention, a reduced rate in frequency of, with potential elimination of, infection is achieved. Thus, in the process of this invention, long term productivity and profitability increase in the operation of a fermentation plant.

It is recognized that individual results at different ethanol fermentation plants operating under different conditions may vary in the relative improvements in the process of this invention in the reduction of acid production and increases in ethanol production.

In the process of this invention, fermentation occurs in a batch or continuous fermentation system. After discharge from the fermentation system, conventional process steps for separation and purification of the ethanol may be performed. The fermentation product may be distilled to separate the ethanol from the bulk of the water present and from the solids (which include inoculant, and grain solids). The solids may be recovered. The distilled ethanol may be further treated, for example by contacting with molecular sieves, to remove remaining water, so that the ethanol product is essentially 100% pure ethanol (200 proof). The purified ethanol is generally treated with a denaturing agent.

A further advantage of the process of this invention is that separation and purification steps including filtration, distillation (heating), and denaturation (addition of a denaturing agent) may be performed in a manner (temperature, selection of denaturing agent) to deactivate any bacteriophage present in the product discharged from the fermentation system and thus, in the finished products. Purified ethanol and grain solids are among the “finished products”. Therefore, release of viable bacteriophages following the completion of the fermentation process can be controlled.

### EXAMPLES

### Example 1

Lactobacillus plantarum is a bacterium commonly isolated from contaminated fuel ethanol fermentation. In this example, a spectrophotometer was used to quantify the growth of L. plantarum in laboratory media. The use of a spectrophotometer allowed rapid determination of bacterial levels by optical density. The optical density indicates the amount of light (at a wavelength of 450 nm) absorbed by a sample. High levels of bacterial growth in a sample well result in higher optical density.

L. plantarum ATCC 8014 was cultured overnight in MRS broth (available from Difco, Sparks, Md.) at 30° C. Stocks of L. plantarum ATCC 8014 bacteriophage (bacteriophage cocktail is referred to in the Examples as “B-8014”) were prepared and titered using the overlay method (Adams, M. H. The Bacteriophages, Interscience Publishers, New York, 1959) with MRS agar. Since bacteriophage (phage) are viruses, they are unable to replicate outside of their bacterial host. Therefore, to enumerate phage, cultures of host bacteria were prepared and mixed with dilutions of phage. These mixtures were placed onto a petri dish containing agar and incubated overnight. The presence of clear zones on “lawns” of bacteria indicated phage infection. These clear zones are known as “plaques” and therefore, phage levels are commonly referred to as plaque forming units per milliliter (PFU/ml).

Overnight cultures of L. plantarum ATCC 8014 were diluted to give approximately 10⁴ CFU/ml (CFU=colony forming units, a standard term used to describe levels of culturable bacteria), which is similar to levels of bacteria commonly found in fuel ethanol fermentation. Samples were prepared in MRS broth consisting of bacteria-phage mixtures of 1 cell to 1 phage (1:1), 10 cells to 1 phage (10:1), 100 cells to one phage (100:1), and 1000 cells to 1 phage (1000:1). In addition to the above combinations, two control samples of L. plantarum alone (i.e., 1 cell to 0 phage) and a second sample consisting of L. plantarum plus 1 part per million (ppm) of Penicillin G (available from Sigma-Aldrich Co., St. Louis, Mo.) were included. Penicillin G is used in the fuel ethanol industry to control bacterial growth.

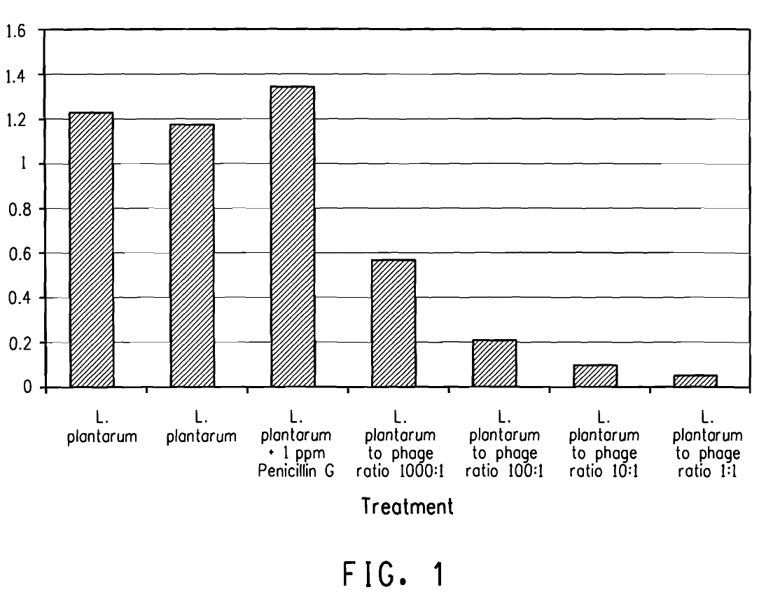

FIG. 1 is a bar graph plotting optical density at 450 nm for presence of Lactobacillus plantarum in a bacteria culture and response to treatment with phage and antibiotic. The first two bars are duplicates illustrating the effect of no treatment. The remaining bars illustrate results of the treatments performed in this Example. As can be seen from FIG. 1, after 40 hours of incubation, the optical density of L. plantarum with no treatment had reached approximately 1.2 at 450 nanometers. The addition of 1 ppm Penicillin G was not effective at reducing the growth of L. plantarum. In fact the optical density of wells containing Penicillin G was higher than for control wells containing L. plantarum only.

The addition of phage B-8014 to the culture of L. plantarum resulted in inhibition of growth as can be seen by the reduction in optical density as seen in FIG. 1. At a concentration of 1000 bacteria to 1 phage, the optical density was reduced to below 0.6 after incubation. At 100 bacteria to 1 phage, the optical density was further reduced to approximately 0.2. Similar results were obtained with 10 bacterial cells per phage. At a level of 1 cell of L. plantarum to one B-8014 phage, bacterial growth was inhibited almost completely (optical density <0.1).

Results indicate that the growth of L. plantarum can be inhibited by exposure to L. plantarum bacteriophage with improved results in comparison to use of Penicillin G, an antibiotic that has been used to treatment bacterial infection in fermentation systems.

### Example 2

Samples of corn mash were collected from a commercial fuel ethanol plant. The corn mash was collected immediately after liquefaction, prior to entry into fermentation. Corn mash samples were frozen upon arrival and defrosted for use in experiments.

Lactobacillus plantarum ATCC 8014 was cultured overnight in MRS broth (Difco, Sparks, Md.). Samples of corn mash were inoculated with L. plantarum at approximately 10⁴ CFU/ml. This level of bacterial contamination is common in fuel ethanol plants. L. plantarum bacteriophage (B-8014) were prepared and titered using the overlay method on MRS agar (as described in Example 1). Corn mash samples containing L. plantarum were treated as indicated in Table 1. One sample of corn mash received no treatment. A second sample was treated by the addition of 3 parts per million (ppm) of Penicillin G. Three additional samples were treated with phage at a bacteria to phage ratio of 1:1, 1:10, and 1:100. All mash samples were incubated at 33° C., the temperature at which commercial fermentation takes place.

The level of bacteria remaining in the test samples was determined by decimal dilutions in phosphate-buffered saline followed by plating of these dilutions onto plates of MRS agar. Plates were incubated overnight at 33° C. and colonies were enumerated. Results are transformed into logarithmic values for comparison in Table 1.

As can be seen in Table 1, each sample initially had a level of approximately 4.76 CFU/ml at time 0 of the experiment. At 24 hours, the control sample grew to 9.06 log CFU/ml. Similar levels were observed at 40 hours for the control. The addition of 3 ppm Penicillin G to a second sample did not affect or control growth of L. plantarum. The addition of phage at a level of 1 phage per bacterial cell or 10 phage per cell had negligible effect on the growth of L. plantarum. When L. plantarum phage were added at a level of 100 phage per bacterial cell, the growth of L. plantarum declined significantly at 24 hours compared to the control, indicating control of growth of L. plantarum by the addition of L. plantarum bacteriophage.

TABLE 1
Response of Lactobacillus plantarum ATCC 8014 in corn mash to
treatment with Penicillin G or Lactobacillus plantarum
B-8014 bacteriophage

| | Log CFU/ml | Log CFU/ml | Log CFU/ml |
|---|---|---|---|
| Treatment | at 0 hours | at 24 hours | at 40 hours |
| L. plantarum (control) | 4.86 | 9.06 | 9.08 |
| L. plantarum + 3 ppm | 4.86 | 9.09 | 9.19 |
| Penicillin G | | | |
| L. plantarum to phage | 4.52 | 8.89 | 9.01 |
| ratio 1:1 | | | |
| L. plantarum to phage | 4.80 | 9.22 | 9.16 |
| ratio 1:10 | | | |
| L. plantarum to phage | 4.73 | 7.21 | 8.63 |
| ratio 1:100 | | | |

As shown by the Examples, it is desirable to prevent and control growth of bacteria in a fermentation system. In particular, adding a bacteriophage cocktail to a fermentation system at a relatively high ratio of phage to bacteria, that is, to use the phage as a preventative, can control and reduce growth of bacteria in the system, where low bacteria concentration is indicated by acetic acid concentration in the system of no greater than 0.3% (weight/volume) and lactic acid concentration of no greater than 0.80% (weight/volume).

What is claimed is:

1. A process to substantially prevent the growth of bacteria in a fermentation system comprising introducing a fermentable material comprising a fermentable sugar and an inoculant into a fermentation system and introducing a cocktail comprising one or more lytic bacteriophage is added to one or more of the fermentable material, the inoculant, or the fermentation system wherein one or more lytic bacteriophage is active against lactic acid bacteria and wherein the cocktail is added in an effective amount at acid concentrations in the fermentation system of acetic acid no greater than 0.30% (weight/volume) and lactic acid no greater than 0.80% (weight/volume).

2. The process of claim 1 the lactic acid bacteria is selected from the group consisting of Lactobacillus, Lactococcus, Enterococcus, Weissella, Leuconostoc, Pediococcus, Streptococcus, Oenococcus and combinations of two or more thereof.

3. The process of claim 1 wherein the amount of the bacteriophage is at least 10 bacteriophage plaque forming units to 1 bacterial colony forming unit.

4. The process of claim 3 wherein the amount of the bacteriophage is at least 100 bacteriophage plaque forming units to 1 bacterial colony forming unit.

5. The process of claim 1 wherein the amount of the bacteriophage is at least 1000 bacteriophage plaque forming units to 1 bacterial colony forming unit.

6. The process of claim 1 wherein the bacteriophage cocktail is added to the fermentable material.

7. The process of claim 1 wherein the bacteriophage cocktail is added to the inoculant.

8. The process of claim 1 wherein the bacteriophage cocktail is added to the fermentation system.

9. The process of claim 1 wherein the fermentable material is derived from corn, wood chips, wheat straw, corn stover, switch grass, and combinations of two or more thereof.

10. The process of claim 9 wherein the fermentable material is derived from corn.

11. The process of claim 10 wherein the bacteriophage is added in an amount of at least 10⁴ plaque forming units per gram of fermentation material.

12. The process of claim 1 wherein the fermentable material is derived from a feedstock selected from the group consisting of milo, barley, millet, sorghum, sugar cane, sugar beets, molasses, whey, potatoes, and combinations of two or more thereof.

13. The process of claim 1 wherein the inoculant is yeast.

14. The process of claim 1 wherein the fermentable sugar is present at a concentration of about 5 to about 40% (weight/volume), based on the volume of the fermentable material.

15. The process of claim 1 wherein the fermentable sugar is present at a concentration of about 10 to 30% (weight/volume), based on the volume of the fermentable material.

16. The process of claim 2 wherein the fermentable material is derived from corn, and the amount of bacteriophage is at least 100 bacteriophage plaque forming units to 1 bacterial colony forming units.
